(19) United States
(12) Patent Application Publication
(10) Pub. No.: US 2022/0218731 A1
(43) Pub. Date: Jul. 14, 2022

(54) COMBINATION THERAPIES FOR CANCER TREATMENT

(71) Applicant: Clear Creek Bio, Inc., Cambridge, MA (US)

(72) Inventors: Vikram S. Kumar, Boston, MA (US); David P. Hesson, Malvern, PA (US)

(21) Appl. No.: 17/606,204

(22) PCT Filed: May 14, 2020
(86) PCT No.: PCT/US20/32792

§ 371 Date: Oct. 25, 2021

Related U.S. Application Data
(60) Provisional application No. 62/851,291, filed on May 22, 2019.

(51) Int. Cl. A61K 31/7056; A61K 31/47; A61K 31/365; A61K 45/06

(52) U.S. Cl. CPC A61K 31/7056; A61K 45/06; A61K 31/365; A61K 31/47

(57) ABSTRACT

The invention provides combination therapies for the treatment of cancer. The combination therapies include a first agent that alters a metabolic pathway and a second agent that targets the metabolic pathway. The combination therapies are useful for treating cancers having tumor cells with different phenotypes, such as cancers of the blood, breast, colon, and prostate.

### CROSS-REFERENCE TO RELATED APPLICATIONS

This application claims the benefit of, and priority to, U.S. Provisional Patent Application No. 62/851,291, filed May 22, 2019, the contents of which are incorporated by reference.

### FIELD OF THE INVENTION

The invention generally relates to therapeutic compositions and methods for the treatment of cancer.

### BACKGROUND

Each year over 8 million people die worldwide from cancer or cancer-related illnesses. Cancer results from unchecked cell growth, and the unregulated cells invade other parts of the body, hijack nutritional resources, and impair the function of other tissues. Cancer is an indiscriminate killer, and nearly two in five people will be diagnosed with cancer at some point in their lives.

A characteristic of many types of cancer cells is their ability to transition between epithelial and mesenchymal states. In their mesenchymal state, cancer cells acquire migratory and invasive properties that allow the cells to infiltrate other tissues. Conversely, in their epithelial state, cancer cells actively proliferate and adhere to each other to form tumors. The ability to transition between the two states is critical to cancer metastasis and disease progression. Significantly, more than 90% of cancer-related deaths result from metastases rather than primary tumors.

The transitioning of cancer cells between different phenotypic states also impedes cancer treatment. Phenotypically-distinct cancer cells differ in their metabolic properties, and metabolic pathways that are critical to the function of cells in one state may be dispensable for cells in another state. Consequently, therapeutic agents that target a particular metabolic pathway may effectively kill one population or subset of cancer cells but leave another population or subset unscathed. Because many cancer therapies fail to account for the variation in metabolic states of cancer cells, they are unable to completely eradicate cancer cells from a patient's body. Thus, such therapies are inadequate, and millions of people continue to die from cancer each year.

### SUMMARY

The invention provides combination cancer therapies that include a first agent that triggers cancer cells to depend on a metabolic pathway and a second agent that interferes with that pathway. Thus, by providing the first agent to a patient, the therapies ensure the efficacy of the second agent. The therapies of the invention a useful for treating a variety of types of cancer, such as blood cancer, breast cancer, colorectal cancer, and prostate cancer, that are characterized by heterogeneous tumor cells that exist in different phenotypic states with different metabolic properties.

The combination therapies of the invention are particularly useful for treating cancers that include tumor cells capable of making the epithelial-mesenchymal transition (EMT). Whereas cancer cells in the epithelial state are actively proliferating, cancer cells in the mesenchymal state are not dividing and thus are resistant to agents that directly or indirectly block DNA replication.

In certain embodiments of the invention, EMT of cancer cells is prevented by providing an inhibitor of p38 mitogen-activated protein kinases as the first agent. Consequently, all cancer cells are forced into a state of active proliferation that places a high demand on de novo synthesis of nucleotides. By providing an inhibitor of a nucleotide synthesis pathway to cells that have been pre-treated with a p38 inhibitor, killing of all cancer cells can be achieved.

In an aspect, the invention provides methods of treating cancer in a subject by providing a first agent that alters a metabolic pathway in a cancer cell and a second agent that targets the metabolic pathway that has been altered in the cancer cell.

Alteration of the metabolic pathway due to the first agent may be associated with a change in a phenotype or property of the cancer cell. Alteration of the metabolic pathway due to the first agent may be associated with apoptosis, differentiation, epithelial-mesenchymal transition, invasion, metastasis, migration, or proliferation.

The first agent may differentially alter the metabolic pathway in the cancer cell compared to a non-cancerous cell. The first agent may increase or decrease activity in the metabolic pathway more in a cancer cell than in a non-cancerous cell.

The first agent may target a signaling pathway. The first agent may target a mitogen-activated protein kinase pathway, AKT pathway, or phosphoinositide 3-kinase pathway. The first agent may target one or more effectors in a signaling pathway. The first agent may target a pathway that includes p38, enhancer of zeste 2 polycomb repressive complex 2 subunit (EZH2), E12/E47, epidermal growth factor receptor (EGFR), FOXC2, Hippo, kallikrein-related peptidase 6 (KLK6), neuropilin-2 (NRP2), polo-like kinase 1 (PLK1), polycomb group PcG (PRC2), Raf, sex determining region Y-box 2 (SOX2), SNAI1, SNAI2, transforming growth factor beta (TGF-β), TWIST1, and yes-associated protein 1 (YAP1), ZEB1, and/or ZEB2.

The first agent may be an inhibitor of p38. The p38 inhibitor may be AMG-548, asiatic acid, BIRB-796, BMS-582949, doramapimod, LY-2228820, pamapimod, PH-797804, SB-202190, SB-203580, SB-203580, SB-239063, SCIO-323, SCIO-469, SD-169, SKF-86002, TAK-715, VX-702, or VX-745.

The metabolic pathway altered by the first agent may be a nucleotide synthesis pathway. The metabolic pathway may be a pyrimidine synthesis pathway or a purine synthesis pathway.

The second agent may be an inhibitor of dihydroorotate dehydrogenase (DHODH). The DHODH inhibitor may be brequinar, pyrazofurin, leflunomide, teriflunomide, or N-(phosphonacetyl)-L-aspartate (PALA), including analogs, derivatives, prodrugs, micellar formulations, sustained release formulations, and salts of any of the aforementioned agents.

The second agent may be an inhibitor of inosine monophosphate dehydrogenase (IMPDH). The IMPDH inhibitor may be mizoribine, mycophenolic acid, ribavirin, selenazofurin, taribavirin, or tiazofurin, including analogs, derivatives, prodrugs, micellar formulations, sustained release formulations, and salts of any of the aforementioned agents.

The method may include receiving information regarding a measured level of a metabolite in a sample from the subject. The metabolite may be in the metabolic pathway altered by the first agent and targeted by the second agent.

The metabolite may be a metabolite in a pyrimidine synthesis pathway. The metabolite in the pyrimidine synthesis pathway may be N-carbamoylaspartate, dihydroorotate, orotate, orotidine 5′-monophosphate (OMP), or uridine monophoshpate (UMP).

The metabolite may be a metabolite in a purine synthesis pathway. The metabolite in the purine synthesis pathway may be guanosine triphosphate (GTP), inosine monophosphate (IMP), xanthine, or xanthine monophosphate (XMP).

The method may include providing to the subject a xanthine oxidase inhibitor. The xanthine oxidase inhibitor may be allopurinol, oxypurinol, tisopurine, topiroxostat, phytic acid, or myoinositol.

The method may include providing the first agent and the second agent simultaneously or sequentially. The method may include providing the first agent during a first stage and providing the second agent during a second stage. The first stage and the second stage may overlap partially, may overlap completely, or may not overlap at all. The first stage and the second stage may be concurrent. The first stage and the second stage may be separated by a gap during which neither the first agent nor the second agent is provided to the subject. The first stage and the second stage may be sequential. The first stage and the second stage may make up a cycle, and the method may include multiple cycles. For example, the method may include two, three, four, five, six, or more cycles.

The first agent may be provided during a first phase, and the second agent may be provided during a second phase that is initiated after the first phase is initiated. The first phase and the second phase may overlap. The second phase may be initiated immediately after the first phase is completed. The first phase and the second phase may be separated by a gap.

The cancer may be a blood cancer. The blood cancer may be leukemia, lymphoma, or myeloma. The cancer may be breast cancer, cervical cancer, colorectal cancer, esophageal cancer, head and neck cancer, lung cancer, ovarian cancer, pancreatic cancer, or prostate cancer. The cancer may be associated with EMT.

### DETAILED DESCRIPTION

Although most tumors originate from a single cell, the majority of tumors display striking heterogeneity in phenotypic characteristics at the time of clinical diagnosis. For example, tumor cells from a single subject commonly vary in cellular morphology, gene expression, metabolism, and motility. This phenotypic variability reflects diversity in the potential of tumor cells to perform various functions, such as proliferation, metastasis, and angiogenesis, that are critical for cancer progression. The diversification of cancer cells allows the pathological cells to escape the body's internal mechanisms of tumor suppression, such as apoptosis, immune surveillance, and senescence.

The intra-tumoral diversity of cancer cells is a major obstacle to effective treatment of cancer. Tumor cells with different phenotypes often differ in their responsiveness to a particular treatment. Therefore, even treatments that kill the vast majority of cancer cells may leave a small fraction unharmed, and the surviving cells can re-establish tumors. Moreover, tumors arising from cells that are insensitive to a particular therapy are likely to be resistant to that therapy, thus limiting the options available for second-round treatment.

The invention solves the problem of treating heterogeneous populations of cancer cells by providing specific combinations of therapeutic agents. The combinations include a first agent that homes in on a population of cancer cells to increase their reliance on a particular metabolic pathway and a second agent that interferes with that pathway. For example, the invention provides therapeutic combinations for treatment of cancers that include a population of proliferating tumor cells and another population of non-proliferating tumor cells. Such combinations include a first agent, such as a p38 inhibitor, that triggers non-proliferating cells to start proliferating. Consequently, the first agent creates a high demand for nucleotide synthesis among all tumor cells. Such combinations also include a second agent that inhibits a nucleotide synthesis pathway, such as purine synthesis pathway or pyrimidine synthesis pathway. The second agent starves the tumor cells of the components necessary to replicate their genomes and make other nucleic acids. Because all of the tumor cells are in proliferation state as a result of the first agent, the second agent eliminates all tumor cells from the body.

### Tumor Cell Heterogeneity

As indicated above, cancer cells in a subject may display vast phenotypic variability, even when the cells are derived from a single tumor. Much of the heterogeneity is attributable to heritable changes in gene expression due to mutations of DNA sequences or epigenetic alterations. For example, many types of tumor cells display elevated mutation rates and increased mutational burden compared to non-cancerous cells. Silencing of gene expression due to hypermethylation of promoter regions is also frequently observed in cancers.

Diversity in tumor cell populations may also result from non-heritable sources. One prevailing theory on the basis of non-heritable diversity is that tumors include a small fraction of cancer stem cells that are capable of both self-renewal and differentiation into other types of cells that make up the majority of tumor cells. Cancer stem cells are thought to contribute to both blood cancers, such as acute myeloid leukemia, and solid tumors, such as breast carcinomas. Another mechanism for generating non-heritable variability is phenotypic plasticity, in which the phenotype of cancer cells is altered in response to cues in the microenvironment. According to the phenotypic plasticity model, tumor cells do not proceed through a structured series of developmental stages but rather adapt fluidly to their environment. Regardless of the basis of the heterogeneity, phenotypic differences among cancer cells are associated with divergent biological responses to stimuli. For example, populations may differ in their ability to undergo apoptosis, differentiation, invasion, metastasis, migration, and/or proliferation.

One example of phenotypic plurality that is critical to cancer progression is the transition between epithelial and mesenchymal phenotypes. In the epithelial-mesenchymal transition (EMT), epithelial cells lose polarity and cell-cell adhesion and gain migratory and invasive properties to become mesenchymal stem cells, which can differentiate into a variety of cell types. EMT and the reverse process, mesenchymal-epithelial transition (MET), are necessary for development of many tissues and organs during embryogenesis. In cancer cells, however, EMT allows cells to depart from primary tumors, enter the bloodstream, and invade other tissues. The escaped tumor cells then undergo MET to establish new tumors at the sites of metastasis. EMT is associated with disease progression is certain types of breast cancer, cervical cancer, colorectal cancer, esophageal cancer, head and neck cancer, lung cancer, ovarian cancer, pancreatic cancer, and prostate cancer.

### Altering Metabolic Pathways in Cancer Cells

Altering a Metabolic Pathway with a First Agent

The invention recognizes that the intra-tumor heterogeneity of cancer cells stems from differences in the activity of specific metabolic pathways. Therefore, agents that target a particular metabolic pathway may prevent growth, proliferation, or migration of one population of tumor cells but have little or no effect on another population. The invention overcomes this problem by using a combination of therapeutic agents that exert different effects on tumor cells. The combination includes first agent that alters the activity of a metabolic pathway in at least a fraction of the tumor cells so that all of the tumor cells rely on that pathway. For example, the first agent may increase flux through a pathway or the demand for products generated by the pathway in a population of tumor cells in which the pathway was previously less active and/or dispensable. The first agent may alter the metabolic pathway directly or produce a change in the activity of the pathway as a secondary effect. The combination then makes use of a second agent that interferes with that pathway to kill or neutralize all tumor cells.

In combination therapies of the invention, the first agent may trigger a cellular change that alters the activity of the metabolic pathway targeted by the second agent. For example, the first agent may promote or inhibit apoptosis, differentiation, epithelial-mesenchymal transition, invasion, metastasis, migration, or proliferation. The first agent may alter the metabolic pathway preferentially in cancer cells or a population of cancer cells compared to non-cancerous cells.

The first agent may alter the activity of a metabolic pathway without targeting the pathway directly. For example, the first agent may target a signaling pathway and exert an effect that alters the metabolic pathway targeted by the second agent.

One example of a signaling pathway that can be targeted by the first agent is the p38 mitogen-activated protein kinase (MAPK)/extracellular-regulated kinase (ERK) pathway. In response to external stimuli, the p38 pathway triggers changes in nuclear gene expression via a series of phosphorylation events. The transcription factor Forkhead box C2 (FOXC2) is a p38 substrate that is critical in cancer progression. FOXC2 is overexpressed in tumor cells from a variety of cancers, including breast cancer, prostate cancer, colorectal cancer, and esophageal cancer, and is required for metastasis of breast cancer cells to the lung. FOXC2 is a key downstream effector of EMT, and FOXC2 activity promotes metastasis of certain types of cancer cells. Inhibition of p38 leads to reduced levels of FOXC2 in breast cancer cells and inhibits their metastasis but does not abate growth of primary tumors.

Without wishing to be bound by a particular theory, it is believed that treatment with a p38 inhibitor increases the homogeneity of tumor cells in a subject by forcing all cells into a proliferative state. In particular, p38 inhibition may eliminate a population of tumor cells having a mesenchymal phenotype that promotes metastasis or shift such a population into an epithelial phenotype that favors proliferation. Therefore, by creating a homogeneous population of proliferating tumor cells, p38 inhibition renders all tumor cells susceptible to agents that interference with nucleic synthesis, such as inhibitors of nucleotide synthesis and other anti-neoplastic agents. Thus, in certain embodiments, the combination therapies of the invention include a p38 inhibitor as a first agent and a nucleotide synthesis inhibitor as a second agent.

It will be understood by one of skill in the art that the exemplary combination described above is for illustrative purposes only and does not limit the scope of the invention. On the contrary, the invention encompasses any combination of agents in which a first agent alters the dependence of tumor cells or a population or subset of tumor cells on a metabolic pathway and a second agent targets that metabolic pathway. In certain embodiments, the first agent increases the dependence of tumor cells or a population or subset of tumor cells on a metabolic pathway so that all of the tumor cells are sensitive to perturbations of that pathway, and the second agent inhibits, disrupts, or interferes with that pathway.

The first agent may target one or more other pathways associated with EMT and/or MET in cancer cells. In addition to the p38 MAPK pathway, other pathways that are associated with EMT and/or MET are pathways that include one or more of the following effectors: enhancer of zeste 2 polycomb repressive complex 2 subunit (EZH2), E12/E47, epidermal growth factor receptor (EGFR), FOXC2, Hippo, kallikrein-related peptidase 6 (KLK6), neuropilin-2 (NRP2), polo-like kinase 1 (PLK1), polycomb group PcG (PRC2), Raf, sex determining region Y-box 2 (SOX2), SNAI1, SNAI2, transforming growth factor beta (TGF-β), TWIST1, yes-associated protein 1 (YAP1), ZEB1, and ZEB2. Signaling pathways involved in EMT and/or MET in cancer cells are described in more detail in, for example, Thierauf J., Veit J. A., Hess J. Epithelial-to-Mesenchymal Transition in the Pathogenesis and Therapy of Head and Neck Cancer, Cancers, 2017;9 doi: 10.3390/cancers9070076; Fedele M., Cerchia L., Chiappetta G. The Epithelial-to-Mesenchymal Transition in Breast Cancer: Focus on Basal-Like Carcinomas, Cancers, 2017;9 doi: 10.3390/cancers9100134; Klymenko Y., Kim O., Stack M.S. Complex Determinants of Epithelial: Mesenchymal Phenotypic Plasticity in Ovarian Cancer, Cancers, 2017;9 doi: 10.3390/cancers9080104; Legras A., Pécuchet N., Imbeaud S., Pallier K., Didelot A., Roussel H., Gibault L., Fabre E., Le Pimpec-Barthes F., Laurent-Puig P., Blons H. Epithelial-to-Mesenchymal Transition and MicroRNAs in Lung Cancer, Cancers, 2017;9 doi: 10.3390/cancers9080101; Gaianigo N., Melisi D., Carbone C. EMT and Treatment Resistance in Pancreatic Cancer, Cancers, 2017;9 doi: 10.3390/cancers9090122; Vu T., Datta P.K. Regulation of EMT in Colorectal Cancer: A Culprit in Metastasis, Cancers, 2017;9 doi: 10.3390/cancers9120171; Grelet S., McShane A., Geslain R., Howe P.H. Pleiotropic Roles of Non-Coding RNAs in TGF-β-Mediated Epithelial-Mesenchymal Transition and Their Functions in Tumor Progression, Cancers, 2017;9 doi: 10.3390/cancers9070075; Roche J., Gemmill R.M., Drabkin H. A. Epigenetic Regulation of the Epithelial to Mesenchymal Transition in Lung Cancer, Cancers, 2017;9 doi: 10.3390/cancers9070072; Fu Z., Wen D. The Emerging Role of Polo-Like Kinase 1 in Epithelial-Mesenchymal Transition and Tumor Metastasis, Cancers, 2017;9 doi: 10.3390/cancers9100131; Blackwell R. H., Foreman K. E., Gupta G. N. The Role of Cancer-Derived Exosomes in Tumorigenicity & Epithelial-to-Mesenchymal Transition, Cancers, 2017;9 doi: 10.3390/cancers9080105; Lu J., Shenoy A. K. Epithelial-to-Pericyte Transition in Cancer, Cancers, 2017;9 doi: 10.3390/cancers9070077, the contents of each of which are incorporated herein by reference.

The first agent may target any signaling pathway or component in a signaling pathway associated with a phenotype or phenotypic variability in cancer cells or a population of cancer cells. For example, modes of tumor cell movement are regulated by Rac and Rho pathways. High Rac activity promotes mesenchymal-type movement, characterized by elongated cellular morphology and dependent on extracellular proteolysis. In contrast, Rho kinase signaling inactivates Rac to stimulate amoeboid movement, in which cells have a rounded morphology and are less dependent on extracellular proteolysis. A first agent may shift tumor cells toward either the mesenchymal or amoeboid phenotypes to render the cells more sensitive to a second agent.

As another example, variations in expression of TRAIL ligand influence the timing of apoptotic response of some cancer cells. A first agent may alter signaling through this pathway to render cancer cells more sensitive to pro-apoptotic signals.

Another signaling pathway that may be targeted by the first agent is the Bcr-Abl pathway. In normal cells, Bcr-Abl activity leads to increased expression, an iron transporter, and iron depletion leads to apoptosis. In leukemias driven by the Bcr-Abl mutation, however, the ligand for 24p3 is down-regulated, and the cancer cells avoid apoptosis. Agents that target this pathway may render leukemia cells more sensitive to pro-apoptotic signals.

The first agent may be an activator, inhibitor, or altered form of a component in the p38 pathway. The first agent may be an inhibitor of p38. For example and without limitation, the p38 inhibitor may be AMG-548, asiatic acid, BIRB-796, BMS-582949, doramapimod, LY-2228820, pamapimod, PH-797804, SB-202190, SB-203580, SB-239063, SCIO-323, SCIO-469, SD-169, SKF-86002, TAK-715, VX-702, or VX-745. Other p38 inhibitors are described in, for example, U.S. Pat. Nos. 5,945,418; 6,093,742; 6,410,540; 6,509,363; 6,528,508; 6,617,324; 6,632,945; 6,635,644; 7,125,898; 7,135,575; 7,169,779; 7,423,047; 7,425,555; 7,521,447; 7,642,276; and 8,410,160; U.S Publication No. 20020065296; and International Publication No. WO1999032110.

Targeting a Metabolic Pathway with a Second Agent

The metabolic pathway altered by the first agent may be a nucleotide synthesis pathway. Tumors typically include a population of cells that are actively proliferating, and proliferation places increased demand on nucleotide synthesis pathways. Consequently, such pathways are suitable targets for the second agent in combination therapies of the invention. Nucleotide synthesis pathways include both pyrimidine synthesis pathways and purine synthesis pathways.

Pyrimidine biosynthesis involves a sequence of enzymatic reactions that result in the conversion of glutamine to uridine monophosphate, as shown below:

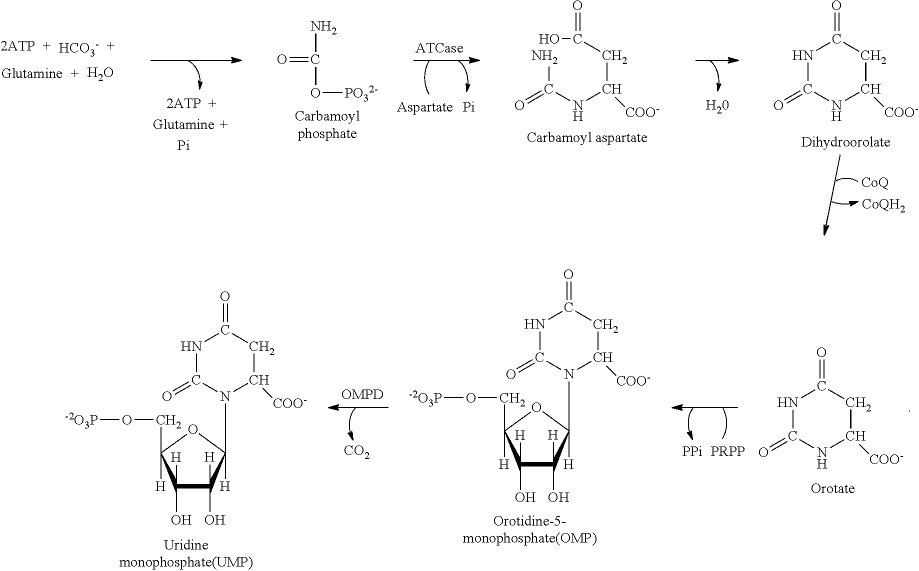

Several of the enzymes in the pyridine synthesis pathway are targets of drugs or drug candidates. For example, aspartate carbamoyltransferase (also known as aspartate transcarbamoylase or ATCase), which catalyzes the conversion of carbamoyl phosphate to carbamoyl aspartate, is inhibited by PALA (N-phosphoacetyl-L-aspartate); dihydroorotate dehydrogenase (DHODH), which catalyzes conversion of dihydroorotate (DHO) to orotate, is inhibited by brequinar, leflunomide, and teriflunomide; and orotidine monophosphate decarboxylase (OMPD), which catalyzes conversion of orotidine monophosphate (OMP) to uridine monophosphate (UMP), is inhibited by pyrazofurin. Therefore, any of the aforementioned agents may be used in combination therapies of the invention.

Purine synthesis pathways include guanine nucleotide synthesis pathways. Some of the key reactions involved in synthesis of guanine nucleotides are shown below:

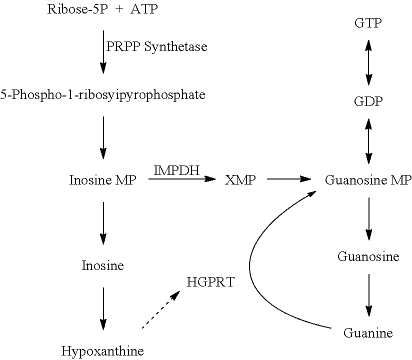

The multi-step conversion of ribose-5-phosphate (ribose-5P) to inosine monophosphate shown in the upper left portion of the diagram is required for all de novo purine synthesis. For synthesis of guanine nucleotides, such guanosine triphosphate (GTP) and deoxyguanosine triphosphate (dGTP), inosine-5′-monophosphate dehydrogenase (IMPDH) catalyzes the nicotinamide adenine dinucleotide (NAD⁺)-dependent oxidation of inosine monophosphate (IMP) to xanthosine monophosphate (XMP). Guanosine monophosphate synthetase (GMPS) then converts XMP to guanosine monophosphate. For synthesis of GTP, shown in the upper right portion of the diagram, two more phosphate moieties are added stepwise to create guanosine diphosphate (GDP) first and then GTP. For synthesis of dGTP, a building block for DNA replication, GDP is converted to deoxyguanosine diphosphate (dGDP), which is then phosphorylated to produce dGTP.

Purines can also be synthesized from the components of degraded macromolecules via salvage pathways. The lower half of the diagram shows that IMP and GMP can be broken down to hypoxanthine and guanine, respectively, via multiple steps. However, hypoxanthine-guanine phosphoribosyltransferase (HGPRT) reverses both series of reactions to regenerate IMP and GMP under appropriate conditions. Thus, HGPRT bypasses IMPDH to synthesize guanine nucleotides via the guanine salvage pathway. Certain tissues and organs are unable to undergo de novo synthesis of purines and rely exclusively on salvage pathways to supply needed nucleotides.

Hypoxanthine can be sequentially converted by xanthine oxidase (XO) first to xanthine and then to uric acid. Inhibition of IMPDH can therefore lead to a high concentration of uric acid in the blood, which may cause medical problems, including kidney stones, gout, and diabetes. Consequently, the therapeutic use of IMPDH inhibitors is usually accompanied by administration of an inhibitor of XO to prevent accumulation of uric acid in the body. Also, hypoxanthine inhibits HGPRT, so the administration of XO increases levels of hypoxanthine to potentiate this blockade of guanine salvage.

Humans have two IMPDH isozymes that have similar enzymatic characteristics. The differential roles of the two isozymes are not well understood.

Several enzymes involved in purine biosynthesis are also of therapeutic interest. For example, IMPDH) is inhibited by tiazofurin; HGPRT is inhibited by 6-mercaptopurine, 1,3-dinitroadamantane, acyclovir, and pentamidine; and dihydrofolate reductase (DHFR), which reduces dihydrofolic acid to tetrahydrofolic acid in the purine salvage pathway, is inhibited by methotrexate. Therefore, any of the aforementioned agents may be used in combination therapies of the invention.

Some agents that may be used in embodiments of the invention are described below.

Brequinar, 6-fluoro-2-(2′-fluoro-1,1′ biphenyl-4-yl)-3-methyl-4-quinoline carboxylic acid, has the following structure:

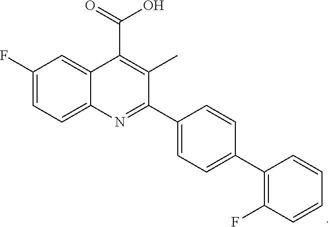 [SMILES: Cc1c(-c2ccc(-c3ccccc3F)cc2)nc2ccc(F)cc2c1C(=O)O]

Brequinar and related compounds are described in, for example, U.S. Pat. Nos. 4,680,299 and 5,523,408, the contents of which are incorporated herein by reference. The use of brequinar to treat leukemia is described in, for example, U.S. Pat. No. 5,032,597 and International Publication No. WO 2017/037022, the contents of which are incorporated herein by reference.

Leflunomide, N-(4′-trifluoromethylphenyl)-5-methylisoxazole-4-carboxamide (I), is described in, for example, U.S. Pat. No. 4,284,786, the contents of which are incorporated herein by reference.

Teriflunomide, 2-cyano-3-hydroxy-N[4-(trifluoromethyl)phenyl]-2-butenamide, is described in, for example, U.S. Pat. No. 5,679,709, the contents of which are incorporated herein by reference.

Pyrazofurin, 5-[(2S,3R,4S,5R)-3,4-dihydroxy-5-(hydroxymethyl)oxolan-2-yl]-4-hydroxy-1H-pyrazole-3-carboxamide, has the following structure:

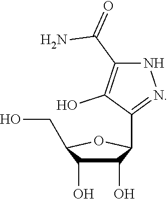

Pyrazofurin and related compounds are described in, for example, U.S. Pat. Nos. 3,674,774 and 3,802,999, the contents of which are incorporated herein by reference.

N-(phosphonacetyl)-L-aspartate (PALA) is described in, for example, Swyryd et al, N-(Phosphonacetyl)-L-Aspartate, a Potent Transition State Analog Inhibitor of Aspartate Transcarbamylase, Blocks Proliferation of Mammalian Cells in Culture, J. Biol. Chem. Vol. 249, No. 21, Issue of November 10, pp. 6945-6950, 1974, the contents of which are incorporated herein by reference.

Tiazofurin, 2-β-D-ribofuranosylthiazole-4-carboxamide, has the following structure:

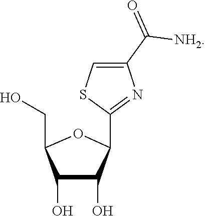

Tiazofurin and methods for making it are known in the art and described in, for example, U.S. Pat. Nos. 4,451,648 and 6,613,896, the contents of which are incorporated herein by reference. Tiazofurin analogs, their activity against tumors, and methods of making them are described in, for example, Popsavin, et al., Synthesis and antiproliferative activity of two new tiazofurin analogues with 2′-amido functionalities, Bioorg. Med. Chem. Lett. 16 (2006) 2773-2776, doi: 10.1016/ibmc1.2006.02.001; and Popsavin, et al., Synthesis and in vitro antitumour activity of tiazofurin analogues with nitrogen functionalities at the C-2′ position, European Journal of Medicinal Chemistry 111 (2016) 114e125. doi: 10.1016/j.ejmech.2016.01.037, the contents of each of which are incorporated herein by reference.

Another IMPDH inhibitor is the non-reversible inhibitor mycophenolic acid. Mycophenolic acid has the following structure:

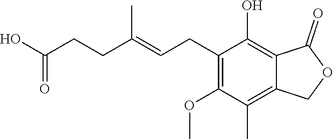 [SMILES: COc1c(C)c2c(c(O)c1C/C=C(\C)CCC(=O)O)C(=O)OC2]

Uses, side effects, and the mechanism of action of mycophenolate are known in the art and described in, for example, Kitchin, J. E., et al., (1997) “Rediscovering mycophenolic acid: A review of its mechanism, side effects, and potential uses” Journal of the American Academy of Dermatology. 37 (3): 445-449. doi:10.1016/S0190-9622(97)70147-6; and Pharmacology North American Edition. Lippincott Williams & Wilkins. 2014, p. 625, ISBN 978-1-4511-9177-6, the contents of each of which are incorporated herein by reference. Salts, derivatives, analogs, and prodrugs of mycophenolic acid are known in the art and described in, for example, Mele T. S., and Halloran, P. F., The use of mycophenolate mofetil in transplant recipients. Immunopharmacology. 2000;47:215-245; International Publication No. WO 2004/096287A2; and U.S. Pat. No. 7,427,636, the contents of each of which are incorporated herein by reference.

Many other inhibitors of IMPDH are known in the art. For example and without limitation, other IMPDH inhibitors include AS2643361, EICAR, FF-10501, mizoribine, mycophenolic acid, ribavirin, selenazofurin, SM-108, taribavirin, VX-148, VX-497, and VX-944. Other IMPDH inhibitors are described in Gebeyehu, G., et al., Ribavirin, Tiazofurin, and Selenazofurin: Mononucleotides and Nicotinamide Adenine Dinucleotide Analogues. Synthesis, Structure, and Interactions with IMP Dehydrogenase, J. Med. Chem. 1985, 28, 99-105; Cuny, G. D., et al., Inosine-5′-monophosphate dehydrogenase (IMPDH) inhibitors: a patent and scientific literature review (2002-2016), Expert Opin Ther Pat., 2017, June 27(6):677-690. doi: 10.1080/13543776.2017.1280463; and U.S. Pat. Nos. 5,807,876; 6,344,465; 6,395,763; 6,399,773; 6,420,403; 6,518,291; 6,541,496; 6,617,323; 6,624,184; 6,653,309; 6,825,224; 6,867,299; 6,919,335; 6,967,214; 7,053,111; 7,060,720; 7,087,642; 7,205,324; 7,329,681; 7,432,290; 7,777,069; and 7,989,498, the contents of each of which are incorporated herein by reference.

### Compositions

The combination therapies of the invention include compositions that contain one or more of the agents described above. The compositions may include one or more agents that alter a metabolic pathway, one or more agents that target the metabolic pathway, or any combination thereof. The one or more agents that alter a metabolic pathway and the one or more agents that target the metabolic pathway may be provided in a single composition or in separate compositions.

In combination therapies of the invention, each agent, such as any of the agents described above, may independently be provided as a prodrug, analog, derivative, salt, or a micellar formulation.

The agents, including prodrugs, analogs, derivatives, and salts thereof, may be provided as pharmaceutical compositions. A pharmaceutical composition may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc.

The tablets may be uncoated, or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. No. 2003/0232877, the contents of each of which are incorporated by reference herein.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Pharmaceutical compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol. sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. The pharmaceutically acceptable carrier may be an encapsulation coating. For example, the encapsulation coating may contain carrageenan, cellulose acetate phthalate, cellulose acetate succinate, cellulose acetate trimellitate, collagen, gelatin, hydroxypropyl methyl cellulose acetate, a methyl acrylate-methacrylic acid copolymer, polyvinyl acetate phthalate shellac, sodium alginate, starch, or zein.

The agents, including prodrugs, analogs, and derivatives thereof, may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Other pharmaceutically acceptable salts may be found in, for example, Remington, The Science and Practice of Pharmacy (20th ed. 2000). Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

### Measuring the Level of a Metabolite in a Sample

The combination therapies of the invention include an agent that targets a metabolic pathway, for example, by stimulating or inhibiting activity of the metabolic pathway. The extent to which a metabolic pathway is stimulated or inhibited is critical for effective cancer treatment: insufficient modulation of the pathway may not kill or block growth of cancer cells, while excessive modulation of the pathway may harm healthy cells. The effect of an agent on a metabolic pathway may be determined by measuring the level of metabolite in the pathway targeted by the agent. Therefore, methods of the invention may include measuring the level of a metabolite in a sample from the subject.

In some embodiments, the metabolite is measured by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), hydrophilic interaction chromatography (HILIC), ultra-performance liquid chromatography (UPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi:10.1002/elps.200500398; Kolch et al., Capillary electrophoresis-mass spectrometry as a powerful tool in clinical diagnosis and biomarker discovery, Mass Spectrom Rev. 24 (6): 959-77. (2005) doi:10.1002/mas.20051; Kanu et al., Ion mobility-mass spectrometry, Journal of Mass Spectrometry, 43 (1): 1-22 (2008) doi:10.1002/jms.1383, the contents of which are incorporated herein by reference.

A sample may be obtained from any organ or tissue in the individual to be tested, provided that the sample is obtained in a liquid form or can be pre-treated to take a liquid form. For example and without limitation, the sample may be a blood sample, a urine sample, a serum sample, a semen sample, a sputum sample, a lymphatic fluid sample, a cerebrospinal fluid sample, a plasma sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, a phlegm sample, a saliva sample, a sweat sample, or a combination of such samples. The sample may also be a solid or semi-solid sample, such as a tissue sample, feces sample, or stool sample, that has been treated to take a liquid form by, for example, homogenization, sonication, pipette trituration, cell lysis etc. For the methods described herein, it is preferred that a sample is from plasma, serum, whole blood, or sputum.

The sample may be kept in a temperature-controlled environment to preserve the stability of the metabolite. For example, DHO is more stable at lower temperatures, and the increased stability facilitates analysis of this metabolite from samples. Thus, samples may be stored at 4° C., −20° C., or −80° C.

In some embodiments, a sample is treated to remove cells or other biological particulates. Methods for removing cells from a blood or other sample are well known in the art and may include e.g., centrifugation, sedimentation, ultrafiltration, immune selection, etc.

The subject may be an animal (such as a mammal, such as a human). The subject may be a pediatric, a newborn, a neonate, an infant, a child, an adolescent, a pre-teen, a teenager, an adult, or an elderly patient. The subject may be in critical care, intensive care, neonatal intensive care, pediatric intensive care, coronary care, cardiothoracic care, surgical intensive care, medical intensive care, long-term intensive care, an operating room, an ambulance, a field hospital, or an out-of-hospital field setting.

The sample may be obtained from an individual before or after administration to the subject of an agent that alters activity of a metabolic pathway, such as inhibitor of an enzyme in the pathway. For example, the sample may be obtained 1 hour, 2 hours, 4 hours, 6 hours, 8 hours, 12 hours, 24 hours, 36 hours, 2 days, 3 days, 4 days, 5 days, 6 days, 7 days or more before administration of an agent, or it may be obtained 1 hour, 2 hours, 4 hours, 6 hours, 8 hours, 12 hours, 24 hours, 36 hours, 2 days, 3 days, 4 days, 5 days, 6 days, 7 days or more after administration of an agent.

### Determining Dosing Regimens

Methods of the invention may include determining a dosing regimen of an agent based on measured levels of a metabolite. Methods of determining dosing regimens of a therapeutic agent based on measured levels of metabolite in a pathway targeted by the agent are known in the art and described in, for example, co-owned, co-pending U.S. application Ser. Nos. 16/364,423, 16/364,436, 16/36,443, and co-owned, co-pending International Application Nos. PCT/US2019/023983 and PCT/US2019/023985, the contents of each of which are incorporated herein by reference. The effect of an agent on its target can best be determined by measuring levels of a metabolite in the targeted pathway, so the choice of metabolite to monitor depends on the agent and target. For example and without limitation, the effect of DHODH inhibitors, such as brequinar, leflunomide, and teriflunomide, can be determined from measured levels DHO; the effect of OMP decarboxylase inhibitors, such as pyrazofurin, can be determined from measured levels of orotate or OMP; and the effect of IMPDH inhibitors, such as mizoribine, mycophenolic acid, ribavirin, selenazofurin, taribavirin, and tiazofurin, can be determined from measured levels of GTP or hypoxanthine.

The dosing regimen may include a dose, i.e., an amount, of the agent that should be administered. The dosing regimen may include a time point for administration of a dose of the agent to the subject. Because the dosing regimen is based on one or more measured levels of a metabolite in a sample obtained from the subject, the dosing regimen is tailored to an individual subject, e.g., a patient. Consequently, the methods of the invention provide customized dosing regimens that account for variability in pharmacokinetic properties, i.e., metabolism of the active pharmaceutical ingredient (API) by the subject, and pharmacodynamics properties, effect of the API on its target, among individuals.

The dosing regimen may be determined by comparing a measured level of a metabolite in a sample obtained from a subject to a reference that provides an association between the measured level and a recommended dosage adjustment of the agent. For example, the reference may provide a relationship between administration of the agent and levels of the metabolite in the subject. The relationship can be empirically determined from a known dose and time of administration of the agent and measured levels of the metabolite at one or more subsequent time points. The reference may include a relationship between measured levels of the agent or a metabolic product of the agent and measured levels of the metabolite.

From the comparison between the measured level of the metabolite and the reference, a dosing regimen may then be determined. The dosing regimen may include a dosage of the agent, a time for administration of the dosage, or both. The dosing regimen may be determined de novo, or it may comprise an adjustment to a previous dosing regimen, such as an adjustment in the dosage, the interval between administration of dosages, or both.

The dosing regimen is designed to deliver the agent to the subject in an amount that achieves a therapeutic effect. The therapeutic effect may be a sign or symptom of the cancer. The therapeutic effect may be inhibition of an enzyme in the metabolic pathway, or it may be a change in an indicator of inhibition of an enzyme in a metabolic pathway. The indicator may be a metabolite in the pathway, and the therapeutic effect may be an increase or decrease in levels of the metabolite. The therapeutic effect may be a decrease in number of cancer cells, a decrease in proliferation of cancer cells, an increase in differentiation of pre-cancerous cells, such as myeloblasts, complete remission of cancer, complete remission with incomplete hematologic recovery, morphologic leukemia-free stat, or partial remission. Increased differentiation of myeloblasts may be assessed by one or more of expression of CD14, expression of CD11b, nuclear morphology, and cytoplasmic granules.

The dosing regimen may ensure that levels of a metabolite are raised or maintained above a minimum threshold required to achieve a certain effect. For example, the dosing regimen may raise or maintain levels of the metabolite above a threshold level in the subject for a certain time period. The time period may include a minimum, a maximum, or both. For example, the dosing regimen may raise or maintain levels of the metabolite above the threshold level for at least 6 hours, 12, hours, 24 hours, at least 48 hours, at least 60 hours, at least 72 hours, at least 84 hours, at least 96 hours, at least 5 days, at least 6 days, at least 7 days, at least 10 days, at least 2 weeks, or more. The dosing regimen may raise or maintain levels of the metabolite above the threshold level for not more than 24 hours, not more than 36 hours, not more than 48 hours, not more than 60 hours, not more than 72 hours, not more than 84 hours, not more than 96 hours, not more than 5 days, not more than 6 days, not more than 7 days, not more than 10 days, or not more than 2 weeks. The dosing regimen may raise or maintain levels of the metabolite above the threshold level for at least 72 hours but not more than 96 hours, for at least 72 hours but not more than 5 days, for at least 72 hours but not more than 6 days, for at least 72 hours but not more than 7 days, for at least 96 hours but not more than 7 days.

The dosing regimen may ensure that levels of a metabolite do not exceed or are maintained below a maximum threshold that is associated with toxicity. Levels of the metabolite above a maximum threshold may indicate that the agent is causing or is likely to cause an adverse event in the subject. For example and without limitation, adverse events include abdominal pain, anemia, anorexia, blood disorders, constipation, diarrhea, dyspepsia, fatigue, fever, granulocytopenia, headache, infection, leukopenia, mucositis, nausea, pain at the injection site, phlebitis, photosensitivity, rash, somnolence, stomatitis, thrombocytopenia, and vomiting.

The dosing regimen may include a time point for administration of one or more subsequent doses to raise or maintain levels of the metabolite above a threshold level for a certain time period. The time point for administration of a subsequent dose may be relative to an earlier time point. For example, the time point for administration of a subsequent dose may be relative to a time point when a previous dose was administered or a time point when a sample was obtained from a subject.

The dosing regimen may include a schedule for administration of doses. For example, doses may be administered at regular intervals, such as every 24 hours, every 36 hours, every 48 hours, every 60 hours, every 72 hours, every 84 hours, every 96 hours, every 5 days, every 6 days, every week, every 2 weeks, every 3 weeks, or every 4 weeks. Alternatively, doses may be administered according to a schedule that does not require precisely regular intervals. For example, doses may be administered once per week, twice per week, three times per week, four times per week, once per month, twice per month, three times per month, four times per month, five times per month, or six times per month.

For example and without limitation, a dosing regimen for administration of a therapeutic agent, such as brequinar, e.g., brequinar sodium, to a human subject may be as follows: 100 mg/m², administered intravenously twice weekly; 125 mg/m², administered intravenously twice weekly; 150 mg/m², administered intravenously twice weekly; 200 mg/m², administered intravenously twice weekly; 250 mg/m², administered intravenously twice weekly; 275 mg/m², administered intravenously twice weekly; 300 mg/m², administered intravenously twice weekly; 350 mg/m², administered intravenously twice weekly; 400 mg/m², administered intravenously twice weekly; 425 mg/m², administered intravenously twice weekly; 450 mg/m², administered intravenously twice weekly; 500 mg/m², administered intravenously twice weekly; 550 mg/m², administered intravenously twice weekly; 600 mg/m², administered intravenously twice weekly; 650 mg/m², administered intravenously twice weekly; 700 mg/m², administered intravenously twice weekly; 750 mg/m², administered intravenously twice weekly; 800 mg/m², administered intravenously twice weekly; 100 mg/m², administered intravenously every 72 hours; 125 mg/m², administered intravenously every 72 hours; 150 mg/m², administered intravenously every 72 hours; 200 mg/m², administered intravenously every 72 hours; 250 mg/m², administered intravenously every 72 hours; 275 mg/m², administered intravenously every 72 hours; 300 mg/m², administered intravenously every 72 hours; 350 mg/m², administered intravenously every 72 hours; 400 mg/m², administered intravenously every 72 hours; 425 mg/m², administered intravenously every 72 hours; 450 mg/m², administered intravenously every 72 hours; 500 mg/m², administered intravenously every 72 hours; 550 mg/m², administered intravenously every 72 hours; 600 mg/m², administered intravenously every 72 hours; 650 mg/m², administered intravenously every 72 hours; 700 mg/m², administered intravenously every 72 hours; 750 mg/m², administered intravenously every 72 hours; 800 mg/m², administered intravenously every 72 hours; 100 mg/m², administered intravenously every 84 hours; 125 mg/m², administered intravenously every 84 hours; 150 mg/m², administered intravenously every 84 hours; 200 mg/m², administered intravenously every 84 hours; 250 mg/m², administered intravenously every 84 hours; 275 mg/m², administered intravenously every 84 hours; 300 mg/m², administered intravenously every 84 hours; 350 mg/m², administered intravenously every 84 hours; 400 mg/m², administered intravenously every 84 hours; 425 mg/m², administered intravenously every 84 hours; 450 mg/m², administered intravenously every 84 hours; 500 mg/m², administered intravenously every 84 hours; 550 mg/m², administered intravenously every 84 hours; 600 mg/m², administered intravenously every 84 hours; 650 mg/m², administered intravenously every 84 hours; 700 mg/m², administered intravenously every 84 hours; 750 mg/m², administered intravenously every 84 hours; 800 mg/m², administered intravenously every 84 hours; 100 mg/m², administered intravenously every 96 hours; 125 mg/m², administered intravenously every 96 hours; 150 mg/m², administered intravenously every 96 hours; 200 mg/m², administered intravenously every 96 hours; 250 mg/m², administered intravenously every 96 hours; 275 mg/m², administered intravenously every 96 hours; 300 mg/m², administered intravenously every 96 hours; 350 mg/m², administered intravenously every 96 hours; 400 mg/m², administered intravenously every 96 hours; 425 mg/m², administered intravenously every 96 hours; 450 mg/m², administered intravenously every 96 hours; 500 mg/m², administered intravenously every 96 hours; 550 mg/m², administered intravenously every 96 hours; 600 mg/m², administered intravenously every 96 hours; 650 mg/m², administered intravenously every 96 hours; 700 mg/m², administered intravenously every 96 hours; 750 mg/m², administered intravenously every 96 hours; 800 mg/m², administered intravenously every 96 hours; 100 mg/m², administered orally twice weekly; 125 mg/m², administered orally twice weekly; 150 mg/m², administered orally twice weekly; 200 mg/m², administered orally twice weekly; 250 mg/m², administered orally twice weekly; 275 mg/m², administered orally twice weekly; 300 mg/m², administered orally twice weekly; 350 mg/m², administered orally twice weekly; 400 mg/m², administered orally twice weekly; 425 mg/m², administered orally twice weekly; 450 mg/m², administered orally twice weekly; 500 mg/m², administered orally twice weekly; 550 mg/m², administered orally twice weekly; 600 mg/m², administered orally twice weekly; 650 mg/m², administered orally twice weekly; 700 mg/m², administered orally twice weekly; 750 mg/m², administered orally twice weekly; 800 mg/m², administered orally twice weekly; 100 mg/m², administered orally every 72 hours; 125 mg/m², administered orally every 72 hours; 150 mg/m², administered orally every 72 hours; 200 mg/m², administered orally every 72 hours; 250 mg/m², administered orally every 72 hours; 275 mg/m², administered orally every 72 hours; 300 mg/m², administered orally every 72 hours; 350 mg/m², administered orally every 72 hours; 400 mg/m², administered orally every 72 hours; 425 mg/m², administered orally every 72 hours; 450 mg/m², administered orally every 72 hours; 500 mg/m², administered orally every 72 hours; 550 mg/m², administered orally every 72 hours; 600 mg/m², administered orally every 72 hours; 650 mg/m², administered orally every 72 hours; 700 mg/m², administered orally every 72 hours; 750 mg/m², administered orally every 72 hours; 800 mg/m², administered orally every 72 hours; 100 mg/m², administered orally every 84 hours; 125 mg/m², administered orally every 84 hours; 150 mg/m², administered orally every 84 hours; 200 mg/m², administered orally every 84 hours; 250 mg/m², administered orally every 84 hours; 275 mg/m², administered orally every 84 hours; 300 mg/m², administered orally every 84 hours; 350 mg/m², administered orally every 84 hours; 400 mg/m², administered orally every 84 hours; 425 mg/m², administered orally every 84 hours; 450 mg/m², administered orally every 84 hours; 500 mg/m², administered orally every 84 hours; 550 mg/m², administered orally every 84 hours; 600 mg/m², administered orally every 84 hours; 650 mg/m², administered orally every 84 hours; 700 mg/m², administered orally every 84 hours; 750 mg/m², administered orally every 84 hours; 800 mg/m², administered orally every 84 hours; 100 mg/m², administered orally every 96 hours; 125 mg/m², administered orally every 96 hours; 150 mg/m², administered orally every 96 hours; 200 mg/m², administered orally every 96 hours; 250 mg/m², administered orally every 96 hours; 275 mg/m², administered orally every 96 hours; 300 mg/m², administered orally every 96 hours; 350 mg/m², administered orally every 96 hours; 400 mg/m², administered orally every 96 hours; 425 mg/m², administered orally every 96 hours; 450 mg/m², administered orally every 96 hours; 500 mg/m², administered orally every 96 hours; 550 mg/m², administered orally every 96 hours; 600 mg/m², administered orally every 96 hours; 650 mg/m², administered orally every 96 hours; 700 mg/m², administered orally every 96 hours; 750 mg/m², administered orally every 96 hours; or 800 mg/m², administered orally every 96 hours.

Minimum and maximum threshold levels of a metabolite depend on a variety of factors, such as the type of subject, metabolite, therapeutic agent, and type of sample. Minimum and maximum threshold levels may be expressed in absolute terms, e.g., in units of concentration, or in relative terms, e.g., in ratios relative to a baseline or reference value. For example, the minimum threshold (below which a patient may receive a dose increase or additional dose) could also be calculated in terms of increase from a pre-treatment level or baseline level of a metabolite.

For example, minimum threshold levels of DHO or orotate in a human plasma sample may be about 0 ng/ml, about 10 ng/mL, about 20 ng/mL, about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1250 ng/ml, about 1500 ng/ml, about 1750 ng/ml, about 2000 ng/ml, about 2500 ng/ml, about 3000 ng/ml, about 3500 ng/ml, about 4000 ng/ml, about 4500 ng/ml, about 5000 ng/ml, about 6000 ng/ml, about 8000 ng/ml, about 10,000 ng/ml, about 12,000 ng/ml, about 15,000 ng/ml, about 20,000 ng/ml, about 25,000 ng/ml, about 30,000 ng/ml, about 40,000 ng/ml, about 50,000 ng/ml, about 75,000 ng/ml, about 100,000 ng/ml, about 150,000 ng/ml, about 200,000 ng/ml, about 300,000 ng/ml, or about 400,000 ng/ml. The minimum threshold may include any value that falls between the values recited above. Thus, the minimum threshold may include any value between 0 ng/ml and 400,000 ng/ml.

Maximum threshold levels of DHO or orotate in a human plasma sample may be about 50 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1250 ng/ml, about 1500 ng/ml, about 1750 ng/ml, about 2000 ng/ml, about 2500 ng/ml, about 3000 ng/ml, about 3500 ng/ml, about 4000 ng/ml, about 4500 ng/ml, about 5000 ng/ml, about 6000 ng/ml, about 8000 ng/ml, about 10,000 ng/ml, about 12,000 ng/ml, about 15,000 ng/ml, about 20,000 ng/ml, about 25,000 ng/ml, about 30,000 ng/ml, about 40,000 ng/ml, about 50,000 ng/ml, about 75,000 ng/ml, about 100,000 ng/ml, about 150,000 ng/ml, about 200,000 ng/ml, about 300,000 ng/ml, about 400,000 ng/ml, or about 500,000 ng/ml. The maximum threshold may include any value that falls between the values recited above. Thus, the maximum threshold may include any value between 50 ng/ml and 500,000 ng/ml.

The minimum threshold of DHO or orotate may be about 1.5 times the baseline level, about 2 times the baseline level, about 2.5 times the baseline level, about 3 times the baseline level, about 4 times the baseline level, about 5 times the baseline level, about 10 times the baseline level, about 20 times the baseline level, about 50 times the baseline level, about 100 times the baseline level, about 200 times the baseline level, about 500 times the baseline level, about 1000 times the baseline level, about 2000 times the baseline level, or about 5000 times the baseline level. The minimum threshold may include any ratio that falls between those recited above. Thus, the minimum threshold may be any ratio between 1.5 times the baseline level and 5000 times the baseline level.

The maximum threshold of DHO or orotate may be about 2 times the baseline level, about 2.5 times the baseline level, about 3 times the baseline level, about 4 times the baseline level, about 5 times the baseline level, about 10 times the baseline level, about 20 times the baseline level, about 50 times the baseline level, about 100 times the baseline level, about 200 times the baseline level, about 500 times the baseline level, about 1000 times the baseline level, about 2000 times the baseline level, about 5000 times the baseline level, or about 10,000 times the baseline level. The maximum threshold may include any ratio that falls between those recited above. Thus, the maximum threshold may be any ratio between 2 times the baseline level and 10,000 times the baseline level.

Dosing of the agent may account for the formulation of the agent. For example, therapeutic agents, such as brequinar, pyrazofurin, leflunomide, teriflunomide, and PALA, may be provided as prodrugs, analogs, derivatives, or salts. Any of the aforementioned chemical forms may be provided in a pharmaceutically acceptable formulation, such as a micellar formulation.

Dosage of the agent also depends on factors such as the type of subject and route of administration. The dosage may fall within a range for a given type of subject and route of administration, or the dosage may adjusted by a specified amount for a given type of subject and route of administration. For example, dosage of brequinar for oral or intravenous administration to a subject, such as a human or mouse, may be about 1 mg/kg, about 2 mg/kg, about 5 mg/kg, about 7.5 mg/kg, about 10 mg/kg, about 15 mg/kg, about 20 mg/kg, about 25 mg/kg, about 50 mg/kg, about 75 mg/kg, or about 100 mg/kg. Dosage of brequinar for oral or intravenous administration to a subject, such as a human or mouse, may be adjusted by about 1 mg/kg, about 2 mg/kg, about 5 mg/kg, about 7.5 mg/kg, about 10 mg/kg, about 15 mg/kg, about 20 mg/kg, about 25 mg/kg, or about 50 mg/kg. Dosage of brequinar for oral or intravenous administration to an animal subject, such as a human or mouse, may be about 50 mg/m², about 100 mg/m², about 200 mg/m², about 300 mg/m², about 350 mg/m², about 400 mg/m², about 500 mg/m², about 600 mg/m², about 700 mg/m², about 750 mg/m², about 800 mg/m², or about 1000 mg/m². Dosage of brequinar for oral or intravenous administration to an animal subject, such as a human or mouse, may be adjusted by about 50 mg/m², about 100 mg/m², about 200 mg/m², about 300 mg/m², about 350 mg/m², or about 400 mg/m².

The methods are useful for providing guidance on dosing of therapeutic agents for individuals. Therefore, the methods may be performed by any party that wishes to provide such guidance. For example and without limitation, the methods may be performed by a clinical laboratory; a physician or other medical professional; a supplier or manufacturer of a therapeutic agent; an organization that provides analytical services to a physician, clinic, hospital, or other medical service provider; or a healthcare consultant.

### Providing Agents

The combination therapies of the invention may include providing the therapeutic agents to a subject. The agents may be provided according to a dosing regimen determined as described above.

Providing the agent to the subject may include administering it to the subject. A dose may be administered as a single unit or in multiple units. The agent may be administered by any suitable means. For example and without limitation, the agent may be administered orally, intravenously, enterally, parenterally, dermally, buccally, topically, transdermally, by injection, intravenously, subcutaneously, nasally, pulmonarily, or with or on an implantable medical device (e.g., stent or drug-eluting stent or balloon equivalents).

In some embodiments, the methods include assessing a metabolite level in a sample from a subject, and determining whether that level is within a threshold range (e.g., above a minimal threshold and/or below a potential toxicity threshold) that warrants dosing, and/or that warrants dosing at a particular level or in a particular amount.

The methods may include administering at least one dose of the agent to a subject whose plasma metabolite level has been determined and is below a pre-determined threshold (e.g., a pre-determined potential toxicity threshold and/or a pre-determined potential efficacy threshold). In some embodiments, the predetermined threshold reflects percent inhibition of a target enzyme in the subject relative to a baseline determined for the subject. In some embodiments, the baseline is determined by an assay.

For example, in some embodiments, in order to maintain inhibition of the target enzyme at an effective threshold, multiple doses of the agent may be administered. In some embodiments, dosing of the agent can occur at different times and in different amounts. The present disclosure encompasses those methods that can maintain inhibition of the target enzyme at a consistent level at or above the efficacy threshold throughout the course of treatment. In some embodiments, the amount of inhibition of the target enzyme is measured by the amount of metabolite in the plasma of a subject.

In some embodiments, more than one dose of the agent is administered to the subject. In some embodiments, the method further comprises a step of re-determining the subject's plasma metabolite level after administration of the at least one dose. In some embodiments, the subject's plasma metabolite level is re-determined after each dose. In some embodiments, the method further comprises administering at least one further dose of the agent after the subject's plasma metabolite level has been determined again (e.g., after administering a first or previous dose), and is below the pre-determined threshold. If the subject's plasma metabolite level is determined to be above a pre-determined threshold, dosing can be discontinued. In some embodiments, therefore, no further dose of the agent is administered until the subject's plasma metabolite level has been determined to again be below a pre-determined threshold.

The methods may include administering an agent to a subject at a dosage level at or near a cell-lethal level. Such dosage can be supplemented with a later dose at a reduced level, or by discontinuing of dosing. For example, in some embodiments, the present disclosure provides a method of administering a dihydroorotate dehydrogenase inhibitor to a subject in need thereof, the method comprising: administering a plurality of doses of an agent, according to a regimen characterized by at least first and second phases, wherein the first phase involves administration of at least one bolus dose of an agent at a cell-lethal level; and the second phase involves either: administration of at least one dose that is lower than the bolus dose; or absence of administration of an agent.

In some embodiments, an agent is not administered during a second phase. In some embodiments, a second phase involves administration of uridine rescue therapy. In some embodiments, a bolus dose is or comprises a cell lethal dose. In some embodiments, a cell lethal dose is an amount of an agent that is sufficient to cause apoptosis in normal (e.g., non-cancerous) cells in addition to target cells (e.g., cancer cells).

In some embodiments, the first phase and the second phase each comprise administering an agent. In some embodiments, the first phase and the second phase are at different times. In some embodiments, the first phase and the second phase are on different days. In some embodiments, the first phase lasts for a period of time that is less than four days. In some embodiments, the first phase comprises administering an agent, followed by a period of time in which no agent is administered. In some embodiments, the period of time in which no agent is administered is 3 to 7 days after the dose during the first phase. In some embodiments, the first phase comprises administering more than one dose.

In some embodiments, an agent is administered during a second phase. In some embodiments, an agent is administered sub-cell-lethal levels during the second phase. In some embodiments, the first phase is repeated after the second phase. In some embodiments, both the first and second phases are repeated.

In some embodiments, the present disclosure provides a method of administering an agent to a subject in need thereof, according to a multi-phase protocol comprising: a first phase in which at least one dose of the agent is administered to the subject; and a second phase in which at least one dose of the agent is administered to the subject, wherein one or more doses administered in the second phase differs in amount and/or timing relative to other doses in its phase as compared with the dose(s) administered in the first phase.

In some embodiments, a metabolite level is determined in a sample from the subject between the first and second phases. In some embodiments, the sample is a plasma sample. In some embodiments, the timing or amount of at least one dose administered after the metabolite level is determined or differs from that of at least one dose administered before the metabolite level was determined.

In some embodiments, the amount of agent that is administered to the patient is adjusted in view of the metabolite level in the subject's plasma. For example, in some embodiments, a first dose is administered in the first phase. In some embodiments, metabolite level is determined at a period of time after administration of the first dose.

In some embodiments, if the metabolite level is below a pre-determined level, the amount of agent administered in a second or subsequent dose is increased and/or the interval between doses is reduced. For example, in some such embodiments, the amount of agent administered may be increased, for example, by 100 mg/m². In some embodiments, the amount of agent administered in a second or subsequent dose is increased by 150 mg/m². In some embodiments, the amount of agent administered in a second or subsequent dose is increased by 200 mg/m². In some embodiments, the amount of agent administered may be increased by an adjustment amount determined based on change in metabolite levels observed between prior doses of different amounts administered to the subject.

In some embodiments, if the metabolite level is above a pre-determined level, the amount of agent administered in a second or subsequent dose is the same as the amount administered in the first or previous dose and/or the interval between doses is the same.

In some embodiments, if the metabolite level is above a pre-determined level, the amount of agent in a second or subsequent dose is decreased and/or the interval between doses is increased. For example, in some such embodiments, the amount of agent administered may be decreased, for example, by 50 mg/m². In some embodiments, if the metabolite level is above a pre-determined level, the amount of agent in a second or subsequent dose is decreased by 75 mg/m². In some embodiments, if the metabolite level is above a pre-determined level, the amount of agent in a second or subsequent dose is decreased by 100 mg/m². In some embodiments, the amount of agent administered may be decreased by an adjustment amount determined based on change in metabolite levels observed between prior doses of different amounts administered to the subject.

In some embodiments, the present disclosure provides a method of administering a later dose of an agent to a patient who has previously received an earlier dose of the agent, wherein the patient has had a level of metabolite assessed subsequent to administration of the earlier dose, and wherein the later dose is different than the earlier dose. The later dose may be different from the earlier dose in amount of agent included in the dose, time interval relative to an immediately prior or immediately subsequent dose, or combinations thereof. The amount of agent in the later dose may be less than that in the earlier dose.

The method may include administering multiple dose of the agent, separated from one another by a time period that is longer than 2 days and shorter than 8 days. For example, the time period may be about 3 days.

In some embodiments, the metabolite level is determined in a sample from the subject before each dose is administered, and dosing is delayed or skipped if the determined metabolite level is above a pre-determined threshold. For example, the metabolite level may be determined about 12 hours, about 24 hours, about 36 hours, about 48 hours, about 60 hours, about 72 hours, about 84 hours, or about 96 hours after administration of an agent

The method may include administering the agent according to a regimen approved in a trial in which a level of metabolite was measured in patients between doses of the agent. The regimen may include multiple doses whose amount and timing were determined in the trial to maintain the metabolite level within a range determined to indicate a degree of target enzyme inhibition below a toxic threshold and above a minimum threshold. The regimen may include determining the metabolite level in the subject after administration of one or more doses of the agent.

In some embodiments, the regimen includes a dosing cycle in which an established pattern of doses is administered over a first period of time. In some embodiments, the regimen comprises a plurality of the dosing cycles. In some embodiments, the regimen includes a rest period during which the agent is not administered between the cycles.

Cancers that can be Treated with Combination Therapies

The combination therapies of the invention are useful for treatment of cancer. In particular, the combination therapies are useful for treating cancers having heterogeneity among tumor cells, as described above.

The cancer may be a blood cancer. For example and without limitation, the cancer may be leukemia, lymphoma, or myeloma.

The cancer may be a cancer of solid tissue. For example and without limitation, the cancer may breast cancer, cervical cancer, colorectal cancer, esophageal cancer, head and neck cancer, lung cancer, ovarian cancer, pancreatic cancer, or prostate cancer. The breast cancer may be basal-like breast cancer, claudin-low breast cancer, or triple-negative breast cancer. The head and neck cancer may be mucosal melanoma and adenoid cystic carcinoma or head and neck squamous cell carcinoma (NHSCC). The lung cancer may be non-small cell lung cancer (NSCLC). The ovarian cancer may be epithelial ovarian cancer. The prostate cancer may be androgen receptor (AR)-negative neuroendocrine (NE)/small-cell prostate cancer.

The cancer may be associated with EMT and/or MET.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of treating a cancer, the method comprising:
providing to a subject having a cancer a first agent that alters a metabolic pathway in a cancer cell; and
providing to the subject a second agent that targets the metabolic pathway that has been altered in the cancer cell.

2. The method of claim 1, wherein alteration of the metabolic pathway due to the first agent is associated with at least one change in the cancer cell selected from the group consisting of apoptosis, differentiation, epithelial-mesenchymal transition, invasion, metastasis, migration, and proliferation.

3. The method of claim 1, wherein the first agent differentially alters the metabolic pathway in the cancer cell compared to a non-cancerous cell.

4. The method of claim 1, wherein the first agent targets a signaling pathway.

5. The method of claim 4, wherein the signaling pathway is a mitogen-activated protein kinase pathway.

6. The method of claim 5, wherein the first agent is an inhibitor of p38.

7. The method of claim 1, wherein the metabolic pathway is a pyrimidine synthesis pathway.

8. The method of claim 7, wherein the second agent is an inhibitor of dihydroorotate dehydrogenase (DHODH).

9. The method of claim 8, wherein the DHODH inhibitor is selected from the group consisting of brequinar, pyrazofurin, leflunomide, teriflunomide, and N-(phosphonacetyl)-L-aspartate (PALA), wherein each of said inhibitors includes analogs, derivatives, prodrugs, micellar formulations, sustained release formulations, and salts thereof

10. The method of claim 7, further comprising receiving information regarding a measured level of a metabolite in the pyrimidine synthesis pathway in a sample from the subject.

11. The method of claim 10, wherein the metabolite is selected from the group consisting of N-carbamoylaspartate, dihydroorotate, orotate, orotidine 5′-monophosphate (OMP), and uridine monophoshpate (UMP).

12. The method of claim 1, wherein the metabolic pathway is a purine synthesis pathway.

13. The method of claim 12, wherein the second agent is an inhibitor of inosine monophosphate dehydrogenase (IMPDH).

14. The method of claim 13, wherein the IMPDH inhibitor is selected from the group consisting of mizoribine, mycophenolic acid, ribavirin, selenazofurin, taribavirin, and tiazofurin, wherein each of said inhibitors includes analogs, derivatives, prodrugs, micellar formulations, sustained release formulations, and salts thereof.

15. The method of claim 12, further comprising receiving information regarding a measured level of a metabolite in the purine synthesis pathway in a sample from the subject.

16. The method of claim 15, wherein the metabolite is selected from the group consisting of guanosine triphosphate (GTP), inosine monophosphate (IMP), xanthine, and xanthine monophosphate (XMP).

17. The method of claim 15, further comprising providing to the subject a xanthine oxidase inhibitor.

18. The method of claim 17, wherein the xanthine oxidase inhibitor is selected from the group consisting of allopurinol, oxypurinol, tisopurine, topiroxostat, phytic acid, and myoinositol.

19. The method of claim 1, wherein the first and second agents are provided sequentially.

20. The method of claim 1, wherein the cancer is a blood cancer.
